(19) United States
(12) Patent Application Publication
(10) Pub. No.: US 2012/0214842 A1
(43) Pub. Date: Aug. 23, 2012

(54) METHODS FOR TREATING DISEASES OF THE RETINA

(75) Inventors: John E. Donello, Dana Point, CA (US); Rong Yang, Mission Viejo, CA (US); Bertrand Leblond, Paris (FR); Eric Beausoleil, Paris (FR); Matthew P. Pando, Paris (FR); Laurent Désiré, Paris (FR); Anne-Sophie Casagrande, Draveil (FR); Veena Viswanath, Irvine, CA (US)

(73) Assignees: ExonHit Therapeutics SA, Paris (FR); ALLERGAN, INC., Irvine, CA (US)

(21) Appl. No.: 13/398,213

(22) Filed: Feb. 16, 2012

Related U.S. Application Data
(60) Provisional application No. 61/444,587, filed on Feb. 18, 2011.
(60) Provisional application No. 61/482,106, filed on May 3, 2011.
(60) Provisional application No. 61/444,602, filed on Feb. 18, 2011.
(60) Provisional application No. 61/482,097, filed on May 3, 2011.

(51) Int. Cl. A61K 31/4709; A61P 35/00; A61P 27/02

(52) U.S. Cl. 514/314

(57) ABSTRACT

Disclosed herein is a method of treating disorders of the retina comprising administering to a patient in need of such treatment a therapeutically effective amount of a compound of Formula I

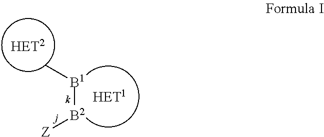

as defined herein. These compounds are useful as PDE10 inhibitors.

### CROSS REFERNCE TO RELATED APPLICATION

The present application claims the benefit of U.S. Provisional Application Ser. Nos. 61/444,587 filed Feb. 18, 2011, 61/482,106 filed May 3, 2011, 61/444,602 filed Feb. 18, 2011, and 61/482,097 filed May 3, 2011, all of which are incorporated by reference herein in their entirety.

### FIELD OF THE INVENTION

The present invention pertains to uses of heteroaromatic compounds that are effective phosphodiesterase (PDE) inhibitors, specifically selective PDE10 inhibitors for treating retinal diseases.

### SUMMARY OF THE INVENTION

The present invention provides a method for treating a disorder of the retina, comprising administering to a patient in need thereof, a therapeutically effective amount of a compound of Formula I:

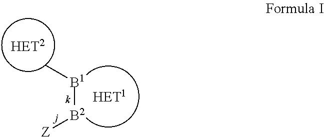

or a pharmaceutically acceptable salt thereof, wherein:

Z is

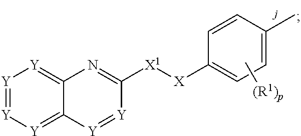

R¹ is each independently selected from a group consisting of hydrogen, halogen, hydroxyl, cyano, C₁ to C₈ alkyl, C₂ to C₈ alkenyl, C₂ to C₈ alkynyl, C₁ to C₈ alkoxy, C₁ to C₈ haloalkyl, C₃ to C₈ cycloalkyl, C₃ to C₈ cycloalkyl-C₁ to C₈ alkyl, 4 to 7 membered heterocycloalkyl, C₁ to C₈ alkylthio, —NR³R³, —O—CF₃, —S(O)ₙ—R³, C(O)—NR³R³, and C₁ to C₈ alkyl substituted with a heteroatom wherein the heteroatom is selected from a group consisting of nitrogen, oxygen and sulfur and wherein the heteroatom may be further substituted with a substituent selected from a group consisting of hydrogen, C₁ to C₈ alkyl, C₃ to C₈ cycloalkyl, C₂ to C₈ alkenyl, C₂ to C₈ alkynyl, and C₁ to C₈ haloalkyl;

each R³ is independently selected from a group consisting of hydrogen, C₁ to C₈ alkyl, C₂ to C₈ alkenyl, C₂ to C₈ alkynyl, C₁ to C₈ haloalkyl, and C₃ to C₈ cycloalkyl;

R² is selected from the group consisting of hydrogen, C₁ to C₈ alkyl, C₃ to C₈ cycloalkyl-C₁ to C₈ alkyl, C₂ to C₈ alkenyl, C₂ to C₈ alkynyl, C₁ to C₈ haloalkyl and C₃ to C₈ cycloalkyl;

HET¹ is selected from a group consisting of a monocyclic heteroaryl and a bicyclic heteroaryl, wherein the monocyclic and bicyclic heteroaryl may be optionally substituted with at least one R⁴;

R⁴ is selected from a group consisting of halogen, hydroxyl, cyano, C₁ to C₈ alkyl, C₂ to C₈ alkenyl, C₂ to C₈ alkynyl, C₁ to C₈ alkoxy, C₃ to C₈ cycloalkyl, C₃ to C₈ cycloalkyl-C₁ to C₈ alkyl, C₁ to C₈ alkylthio, and C₁ to C₈ alkyl substituted with a substituent is selected from the group consisting of —OR⁸, —NR⁸R⁸, and —SR⁸, wherein R⁸ is independently selected from the group consisting of hydrogen and C₁ to C₈ alkyl;

HET² is a monocyclic or bicyclic heteroaryl, wherein the monocyclic and bicyclic heteroaryl optionally substituted with at least one R⁵, with the proviso that HET² is not tetrazole;

R⁵ is independently selected from a group consisting of halogen, hydroxyl, cyano, C₁ to C₈ alkyl, C₂ to C₈ alkenyl, C₂ to C₈ alkynyl, C₁ to C₈ alkoxy, C₃ to C₈ cycloalkyl, C₃ to C₈ cycloalkyl-C₁ to C₈ alkyl, C₁ to C₈ alkylthio, —NR⁷R⁷and C₁ to C₈ haloalkyl;

B¹ and B² are adjacent atoms in Het¹ which are independently selected from a group consisting of carbon and nitrogen;

bond j is a covalent bond between Z and B²;

bond k is a covalent bond in Het¹ between B¹ and B²;

X and X¹ are each independently selected from the group consisting of oxygen, sulfur, C(R₂)₂ and NR₂; provided that at least one of X or X¹ is carbon;

Y is selected from a group consisting of carbon and nitrogen, provided that when Y is carbon it is substituted with R⁶;

wherein each R⁶ is independently selected from a group consisting of hydrogen, halogen, hydroxyl, cyano, C₁ to C₈ alkyl, C₂ to C₈ alkenyl, C₂ to C₈ alkynyl, C₁ to C₈ alkoxy, C₁ to C₈ cycloalkyl, C₃ to C₈ cycloalkyl-C₁ to C₈ alkyl, C₁ to C₈ alkylthio, C₁ to C₈ haloalkyl, —NR⁷R⁷, —O—CF₃, —S(O)m—R⁷, and C(O)—NR⁷R⁷, C₁ to C₈ alkyl substituted with a heteroatom wherein the heteroatom is selected from a group consisting of nitrogen, oxygen and sulfur and wherein the heteroatom may be further substituted with a substituent selected from the group consisting of hydrogen, C₁ to C₈ alkyl, C₃ to C₈ cycloalkyl, C₂ to C₈ alkenyl, C₂ to C₈ alkynyl, and C₁ to C₈ haloalkyl; and

wherein each R⁷ is independently selected from the group consisting of hydrogen and C₁-C₈ alkyl; p is 1, 2 or 3; n is 0, 1 or 2; and m is 0, 1 or 2.

### BRIEF DESCRIPTION OF THE FIGURES

FIG. 1 shows that the compound MP-10 prevents loss of ERG function in an ABCA4/RDH8 mouse model.

FIG. 2 shows that the compound MP-10 protects photoreceptor cell loss in the ABCA4/RDH8 mouse model. Higher grade indicates more loss of photoreceptor cells.

FIG. 3 shows that measurement of autofluorescence by SLO demonstrates that the compound MP-10 reduces the appearance of autofluorescence in the model mice retina.

FIG. 4 shows that PDE10A is expressed in the photoreceptors in human and non-human primates as shown by immunohistochemistry with Anti-human PDE10A Ab. (Abcam 14622-100).

### DETAILED DESCRIPTION OF THE INVENTION

### Conditions of the Retina

The compound of the invention may be used to treat diseases of the retina. By “diseases of the retina,” the applicants mean any condition of the retina which impairs the normal functioning of the retina, its surrounding tissues, or the eye. These include macular degeneration, myopic retinal degeneration, diabetic retinopathy, choroidal neovascularization, macular edema (also referred to as cystoid macular edema and macular swelling), epiretinal membrane (macular pucker), macular hole, retinitis (such as retinitis pigmentosa), macular dystrophies (such as Stargardt's juvenile macular degeneration, Best's vitelliform dystrophy, cone dystrophies, and pattern dystrophy of the retinal pigmented epithelium), retinal detachment, retinal trauma, retinal tumors and retinal diseases associated with them, congenital hypertrophy of the retinal pigmented epithelium, acute posterior multifocal placoid pigment epitheliopathy, acute retinal pigment epithelitis, and uveitis (including iritis, pars planitis, choroiditis, retinitis, and chorioretinitis).

Macular degeneration, also referred to as age-related macular degeneration, is the most common cause of vision loss in the United States in those 50 or older, and its prevalence increases with age. AMD is classified as either wet (neovascular) or dry (non-neovascular). The dry form of the disease is most common. It occurs when the central retina has become distorted, pigmented, or most commonly, thinned, a process associated with atrophy of the retinal pigment epithelium and loss of macular photoreceptors. The result is central geographic atrophy. The wet form of the disease is responsible for most severe loss of vision. The wet form is usually associated with aging, but other diseases that can cause wet macular degeneration include severe myopia and some intraocular infections such as histoplasmosis, which may be exacerbated in individuals with AIDS. The wet form is characterized by abnormal blood vessels growing through the retinal pigment epithelium, resulting in hemorrhage, exudation, scarring, or retinal detachment.

Retinopathy associated with diabetes is a leading cause of blindness in type 1 diabetes, and is also common in type 2 diabetes. The degree of retinopathy depends on the duration of the diabetes, and generally begins to occur ten or more years after onset of diabetes. Diabetic retinopathy may be classified as (1) non-proliferative or background retinopathy, characterized by increased capillary permeability, edema, hemorrhage, microaneurysms, and exudates; or 2) proliferative retinopathy, characterized by neovascularization extending from the retina to the vitreous, scarring, fibrous tissue formation, and potential for retinal detachment. Diabetic retinopathy is believed to be caused, at least in part, by the development of glycosylated proteins due to high blood glucose. Glycosylated proteins generate free radicals, resulting in oxidative tissue damage and depletion of cellular reactive oxygen species (ROS) scavengers, such as glutathione.

In choroidal neovascular membrane, abnormal blood vessels stemming from the choroid grow up through the retinal layers. The fragile new vessels break easily, causing blood and fluid to pool within the layers of the retina.

In macular edema, which can occur as a result of disease, injury or surgery, fluid collects within the layers of the macula, causing blurred, distorted central vision.

Epiretinal membrane is a cellophane-like membrane that forms over the macula, affecting the central vision by causing blur and distortion. As it progresses, the traction of the membrane on the macula may cause swelling. The disease is seen most often in people over 75 years of age.

Retinitis pigmentosa is a retinal degeneration characterized by night blindness and progressive loss of peripheral vision, eventually leading to total blindness; ophthalmoscopic changes include dark mosaic-like retinal pigmentaion, attenuation of the retinal vessels, waxy pallor of the optic disc, and in the advanced forms, macular degeneration. In some cases there can be a lack of pigmentation. Retinitis pigmentosa can be associated to degenerative opacity of the vitreous body, and cataract.

Macular dystrophy is a term applied to a heterogeneous group of diseases that collectively are the cause of severe visual loss in a large number of people. A common characteristic of macular dystrophy is a progressive loss of central vision resulting from the degeneration of photoreceptor cells in the retinal macula. In many forms of macular dystrophy, the end stage of the disease results in legal blindness. More than 20 types of macular dystrophy are known. Some of these are, for example, age-related macular dystrophy, Stargardt-like dominant macular dystrophy, recessive Stargardt's disease, atypical vitelliform macular dystrophy (VMD1), Usher Syndrome Type 1B, autosomal dominant neovascular inflammatory vitreoretinopathy, familial exudative vitreoretinopathy, and Best's macular dystrophy (also known as hereditary macular dystrophy or Best's vitelliform macular dystrophy (VMD2).

Stargardt-like dominant macular dystrophy (also called autosomal dominant macular atrophy) is a juvenile-onset macular degeneration. Patients afflicted with this disease generally have normal vision as young children, but during childhood, visual loss begins, which rapidly progresses to legal blindness. Clinically it is characterized by the presence of an atrophic macular lesion with sharp borders and is often associated with yellow fundus flecks.

Best's macular dystrophy is an inherited autosomal dominant macular dystrophy of unknown biochemical cause. The disease has an age of onset that can range from childhood to after 40. Clinical symptoms include, at early stages, an abnormal accumulation of the yellowish material lipofuscin in the retinal pigmented epithelium (RPE) underlying the macula. This gives rise to a characteristic “egg yolk” appearance of the RPE and gradual loss of visual acuity. With increasing age, the RPE becomes more and more disorganized, as the lipofuscin accumulations disperse and scarring and neovascularization take place. These changes are accompanied by further loss of vision.

The pathological features seen in Stargardt-like dominant macular dystrophy and Best's macular dystrophy are in many ways similar to the features seen in age-related macular dystrophy (AMD), the leading cause of blindness in older patients in the developed world.

Retinal detachment occurs when the sensory layers of the retina become separated from their underlying supporting tissue of retinal pigment epithelium and the choroid. Generally, retinal detachment is caused by a retinal tear or the presence of vitreous traction, either of which may occur spontaneously or may be due to trauma. Retinal detachment may also result from pathology, such as retinopathy of prematurity in premature infants or diabetic retinopathy in diabetic individuals. Symptoms of retinal detachment are painless and sudden segmental or total visual loss in one eye. When there is a tear, or when there is traction causing separation of the retina from its underlying structures, the liquid vitreous passes through the opening and into the subretinal space, inducing further exudation in the subretinal space. The retina gradually separates and detaches from the underlying retinal pigment epithelium. This deprives the outer retina of its normal supply of oxygen and nutrients from the choroid. With time, retinal detachment also results in loss of vision, due to loss of photoreceptor cells located in the outer part of the retina.

By “treat,” the applicants mean to deal with medically. The term includes administering the compound of the invention to alleviate symptoms of a retinal disease, such as the decrease in visual acuity that accompanies macular degeneration, as well as to address the physiological changes associated with the disease, such as the abnormal blood vessel growth that accompanies that condition.

### The Compounds of the Invention

As set forth in the summary of the invention, the present invention provides a compound for Formula I, or a pharmaceutical acceptable salt thereof, for treating retinal diseases.

In another embodiment of the invention, in Formula I, HET¹ is a 5 membered heteroaryl group.

In another embodiment of the invention, in Formula I, HET¹ is selected from the group consisting of pyrazole, isoxazole, triazole, oxazole, thiazole and imidazole.

In another embodiment of the invention, in Formula I, HET² is selected from the group consisting of 4-pyridyl, 4- pyridazine and isoxazole.

In another embodiment of the invention, in Formula I, HET² is 4-pyridyl.

In another embodiment, in Formula I, the compound is selected from the group consisting of:

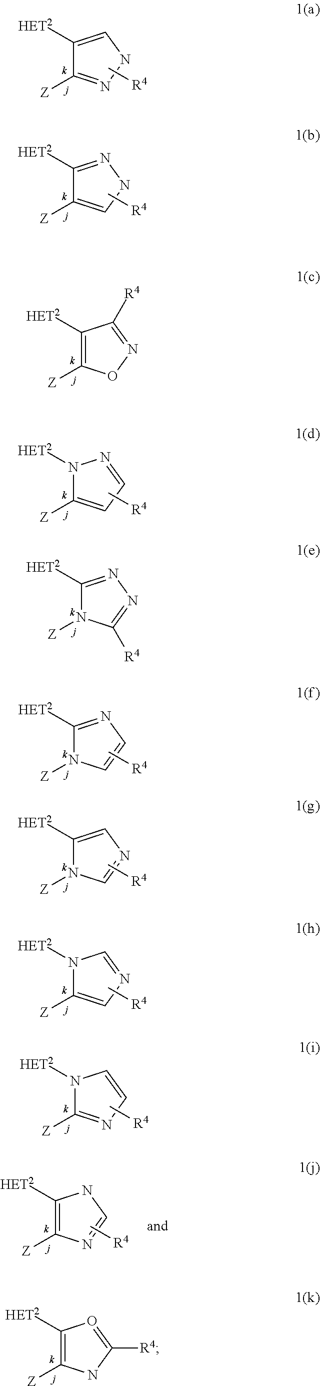

or a pharmaceutically acceptable salt thereof, wherein wherein j, k, Z HET² and R⁴ are as defined in Formula I above.

In another embodiment, the compound of Formula I has the structure

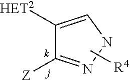

or a pharmaceutically acceptable salt thereof.

In another embodiment, the compound of Formula I has the structure

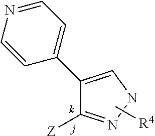

or a pharmaceutically acceptable salt thereof.

In another embodiment of the method of the invention, in Formula I, Y is selected from a group consisting of carbon and nitrogen, provided that not more than one Y is nitrogen.

In another embodiment of the invention, in Formula I, X¹ is carbon and X is oxygen.

In another embodiment of the invention all Y's are carbon (that is, the heteroaryl is quinoline).

In another embodiment, in Formula I, HET¹ is not tetrazole.

In another embodiment, the compound of Formula I is selected from the group consisting of:

2-[-4-4-(4-Pyridin-4-yl-2H-pyrazol-3-yl)-phenoxymethyl]-quinoline;

2-[4-(2-Methyl-4-pyridin-4-yl-2H-pyrazol-3-yl)-phenoxymethyl]-quinoline;

2-[4-(1-Methyl-4-pyridin-4-yl-1H-pyrazol-3-yl)-phenoxymethyl]-quinoline;

2-[4-(2-Ethyl-4-pyridin-4-yl-2H-pyrazol-3-yl)-phenoxymethyl]-quinoline;

2-[4-(1-Ethyl-4-pyridin-4-yl-1H-pyrazol-3-yl)-phenoxymethyl]-quinoline;

Dimethyl-(2-{4-pyridin-4-yl-3-[4-(quinolin-2-ylmethoxy)-phenyl]-pyrazol-1-yl}-ethyl)-amine;

Dimethyl-(2-{4-pyridin-4-yl-5-[4-(quinolin-2-ylmethoxy)-phenyl]-pyrazol-1-yl}-ethyl)-amine;

1-{4-Pyridin-4-yl-3-[4-(quinolin-2-ylmethoxy)-phenyl]-pyrazol-1-yl}-propan-2-ol;

1-{4-Pyridin-4-yl-5-[4-(quinolin-2-ylmethoxy)-phenyl]-pyrazol-1-yl}-propan-2-ol;

2-[4-(2-Isopropyl-4-pyridin-4-yl-2H-pyrazol-3-yl)-phenoxymethyl]-quinoline;

2-[4-(4-Pyridin-4yl-isoxazol-5-yl)-phenoxymethyl]-quinoline;

2-[4-(5-Pyridin-4-yl-pyrimidin-4-yl)-phenoxymethyl]-quinoline;

2-[4-(2-Methyl-5-pyridin-4-yl-pyrimidin-4-yl)-phenoxymethyl]-quinoline;

2-[4-(2-Methyl-6-pyridin-4-yl-pyrazolo[1,5-a]pyrimidin-7-yl)-phenoxymethyl]-quinolin

2-[4-(2-Methyl-6-pyridin-4-yl-[1,2,4]triazolo[1.5-a]pyrimidin-7-yl)-phenoxymethyl]-quinoline 2-[4-(4-Pyridazin-4-yl-2H-pyrazol-3-yl)-phenoxymethyl]-quinoline;

2-[4-(1-Methyl-4-pyridazin-4-yl-1H-pyrazol-3-yl)-phenoxymethyl]-quinoline;

2-[4-(2-Methyl-4-pyridazin-4-yl-2H-pyrazol-3-yl)-phenoxymethyl]-quinoline;

2-[-4-(4-Pyrimidin-4yl-2H-pyrazol-3-yl)-phenoxymethyl]-quinoline;

2-[4-(4-Pyridazin-3-yl-2H-pyrazol-3-yl)-phenoxymethyl]-quinoline;

2-{4-[4-(3-Methyl-isoxazol-5-yl)-2H-pyrazol-3-yl]-phenoxymethyl}-quinoline;

2-{4-[2-Methyl-4-(3-methyl-isoxazol-5-yl)-2H-pyrazol-3-yl]-phenoxymethyl}-quinolin

2-{4-[1-Methyl-4-(3-methyl-isoxazol-5-yl)-1H-pyrazol-3-yl]-phenoxymethyl}-quinolin

2-{4-[2-Methyl-5-(3-methyl-isoxazol-5-yl)-pyrimidin-4-yl]-phenoxymethyl}-quinoline;

2-[4-(2-Pyridin-4-yl-2H-pyrazol-3-yl)-phenoxymethyl]-quinoline;

2-[4-(3-Methyl-5-pyridin-4-yl[1,2,4]triazol-4-yl)-phenoxymethyl]-quinoline;

2-[4-(1-Methyl-4-pyridin-4-yl-1H-pyrazol-3-yl)-phenoxymethyl]-quinoxaline

7-Chloro-2-[4-(1-methyl-4-pyridin-4-yl-1H-pyrazol-3-yl)-phenoxymethyl]-quinoline hydrogen chloride;

6-Fluoro-2-[4-(1-methyl-4-pyridin-4-yl-1H-pyrazol-3-yl)-phenoxymethyl]-quinoline hydrogen chloride;

2-[2-Fluoro-4-(4-pyridin-4-yl-1H-pyrazol-3-yl)-phenoxymethyl]-quinoline;

2-[2-Fluoro-4-(1-methyl-4-pyridin-4-yl-1H-pyrazol-3-yl)-phenoxymethyl]-quinoline;

2-[2,3-Difluoro-4-(1-methyl-4-pyridin-4-yl-1H-pyrazol-3-yl)-phenoxymethyl]-quinoline;

2-[3-Fluoro-4-(4-pyridin-4-yl-1H-pyrazol-3-yl)-phenoxymethyl]-quinoline;

2-[4-(5-Pyridin-4-yl-1H-pyrazol-4-yl)-phenoxymethyl]-quinoline;

2-[4-(1-Methyl-5-pyridin-4-yl-1H-pyrazol-4-yl)-phenoxymethyl]-quinoline 20[4-(Methyl-3-pyridin-4-yl-1H-pyrazol-4-yl)-phenoxymethyl]-quinoline;

2-Methyl-1-{4-pyridin-4-yl-3-[4-(quinolin-2-ylmethoxy)-phenyl]-pyrazol-1-yl}-propan-2-ol;

2-Methyl-1-{4-pyridin-4-yl-5-[4-(quinolin-2-ylmethoxy)-phenyl]-pyrazol-1-yl}-propan-2-ol;

(R)-1-{4-Pyridin-4-yl-3-[4-(quinolin-2-ylmethoxy)-phenyl]-pyrazol-1-yl}-propan-2-ol;

(S)-1-{4-Pyridin-4-yl-3-[4-(quinolin-2-ylmethoxy)-phenyl]-pyrazol-1-yl}-propan-2-ol;

2-[4-(1-Isopropyl-4-pyridin-4-yl-1H-pyrazol-3-yl)-phenoxymethyl]-quinoline;

2-[4-(1-Isobutyl-4-pyridin-4-yl-1H-pyrazol-3-yl)-phenoxymethyl]-quinoline;

2-[4-(1-Methyl-4-pyridin-4-yl-1H-pyrazol-3-yl)-phenoxymethyl]-[1.8]naphthyridine;

2-{2-[4-(4-Pyridin-4-yl-2H-pyrazol-3-yl)-phenyl]-ethyl}-quinoline;

2-{2-[4-(1-Methyl-4-pyridin-4-yl-1H-pyrazol-3-yl)-phenyl]-ethyl}-quinoline;

2-{4-[4-(2-Chloro-pyridin-4-yl)-1H-pyrazol-3-yl]-phenoxymethyl}-quinoline;

2-{4-[4-(2-Chloro-pyridin-4-yl)-1-methyl-1H-pyrazol-3-yl]-phenoxymethyl}-quinoline;

2-{4-[1-Methyl-4-(2-methyl-pyridin-4-yl)-1H-pyrazol-3-yl]-phenoxymethyl}-quinoline;

Dimethyl-(4-{1-methyl-3-[4-(quinolin-2-ylmethoxy)-phenyl]-1H-pyrazol-4-yl}-pyridin-2-yl)-amine;

2-[4-(5-Pyridin-4-yl-pyrazol-1-yl)-phenoxymethyl]-quinoline;

2-[4-(3-Methyl-5-pyridin-4-yl-pyrazol-1-yl)-phenoxymethyl]-quinoline;

2-[2-Chloro-4-(4-pyridin-4-yl-1H-pyrazol-3-yl)-phenoxymethyl]-quinoline;

2-[2-Chloro-4-(1-methyl-4-pyridin-4-yl-1H-pyrazol-3-yl)-phenoxymethyl]-quinoline;

2-[4-(4-Pyridin-4-yl-4H-[1,2,4]triazol-3-yl)-phenoxymethyl]-quinoline;

2-[4-(5-Pyridin-4-yl-[1,2,4]triazol-1-yl)-phenoxymethyl]-quinoline;

2-[4-(3-Methyl-5-pyridin-4-yl-[1,2,4]triazol-1-yl)-phenoxymethyl]-quinoline;

2-[4-(2-Pyridin-4-yl-2H-[1,2,4]triazol-3-yl)-phenoxymethyl]-quinoline;

2-[4-(5-Methyl-2-pyridin-4-yl-2H-[1,2,4]triazol-3-yl)-phenoxymethyl]-quinoline;

8-Methoxy-2-[4-(1-methyl-4-pyridin-4-yl-1H-pyrazol-3-yl)-phenoxymethyl]-quinoline;

2-[4-(1-Methyl-4-pyridin-4-yl-1H-pyrazol-3-yl)-phenoxymethyl]-pyrido[1,2-a]pyrimidin-4-one;

2-[4-(1-Methyl-4-pyridin-4-yl-1H-pyrazol-3-yl)-phenoxymethyl]-quinazoline;

2-[3-Fluoro-4-(1-methyl-4-pyridin-4-yl-1H-pyrazol-3-yl)-phenoxymethyl]-quinoline;

4-Chloro-2-[4-(1-methyl-4-pyridin-4-yl-1H-pyrazol-3-yl)-phenoxymethyl]-quinoline;

4-Methoxy-2-[4-(1-methyl-4-pyridin-4-yl-1H-pyrazol-3-yl)-phenoxymethyl]-quinoline;

Dimethyl-{2-[4-(1-methyl-4-pyridin-4-yl-1H-pyrazol-3-yl)-phenoxymethyl]-quinolin-4-yl}-amine;

2-[4-(1-Methyl-4-pyridin-4-yl-1H-pyrazol-3-yl)-benzyloxy]-quinoline di-succinic acid;

2-((4-(5-(pyridin-4-yl)oxazol-4-yl)phenoxy)methyl)quinoline;

2-((4-(2-methyl-5-(pyridin-4-yl)oxazol-4-yl)phenoxy)methyl)quinoline;

2-((4-(3-Methyl-4-(pyridin-4-yl)-1H-pyrazol-5-yl)phenoxy)methyl)quinoline;.

2-((4-(1,3-dimethyl-4-(pyridin-4-yl)-1H-pyrazol-5-yl)phenoxy)methyl)quinoline;

2-((4-(1,5-dimethyl-4-(pyridin-4-yl)-1H-pyrazol-3-yl)phenoxy)methyl)quinoline;

2-(1-(4-(1-methyl-4-(pyridin-4-yl)-1H-pyrazol-3-yl)phenoxy)ethyl)quinoline;

2-((4-(5-(pyridin-4-yl)-1,2,3-triazol-4-yl)phenoxy)methyl)quinoline;

2-((4-(2-methyl-5-(pyridin-4-yl)-2H-1,2,3-triazol-4-yl)phenoxy)methyl)quinoline;

2-((4-(3-methyl-5-(pyridin-4-yl)-3H-1,2,3-triazol-4-yl)phenoxy)methyl)quinoline;

2-((4-(1-(pyridin-4-yl)-1H-imidazol-2-yl)phenoxy)methyl)quinoline;

2-((4-(5-(pyridin-4-yl)-1H-imidazol-1-yl)phenoxy)methyl)quinoline;

2-((4-(2-methyl-5-(pyridin-4-yl)-1H-imidazol-1-yl)phenoxy)methyl)quinoline;

2-((4-(2-ethyl-5-(pyridin-4-yl)-1H-imidazol-1-yl)phenoxy)methyl)quinoline;

2-((4-(2-(pyridin-4-yl)-1H-imidazol-1-yl)phenoxy)methyl)quinoline;

and pharmecutical acceptable salts thereof.

In another embodiment, the compound of Formula I is selected from the group consisting of:

2-{4-[Pyridin-4-yl-2-(2,2,2-trifluoro-ethyl)-2H-pyrazol-3-yl]-phenoxymethyl}-quinoline:

2-{4-[Pyridin-4-yl-1-(2,2,2-trifluoro-ethyl)-1H-pyrazol-3yl]-phenoxymethyl}-quinoline;

2-{3-Fluoro-4-[4-pyridin-4-yl-1-(2,2,2-trifluoro-ethyl)-1H-pyrazol-3yl]-phenoxymethyl}-quinoline;

2-{3-Fluoro-4-[4-pyridin-4-yl-1-(2,2,2-trifluoro-ethyl)-1H-pyrazol-3-yl]-phenoxymethyl}-quinoxaline;

2-{4-[4-Pyridin-4-yl-1-(2,2,2-trifluoro-ethyl)1H-pyrazol-3yl]-phenoxymethyl}-quinoxaline;

and pharmaceutically acceptable salts thereof.

The above compounds are described in WO 2006/072828 A2.

In one preferred embodiment, the compound of Formula I is administered as the succinate salt.

In another preferred embodiment, the compound of Formula I has the structure

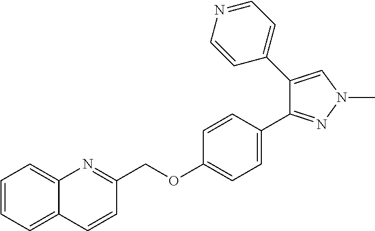

or a pharmaceutically acceptable salt thereof, and in one embodiment, the succinate salt of the compound.

The chemical name of this compound (free base form) is 2-[4-(1-methyl-4-pyridin-4-yl-1H-pyrazol-3-yl)-phenoxymethyl]-quinoline. Methods for making this compound, also known as PF-2545920 or MP-10, may be found in U.S. Pat. No. 7,429,665, the contents of which are incorporated by reference in their entirety for all purposes.

The succinate salt of this compound may be prepared as follows, according to U.S. Patent Application Publication No. 2010/063089, the contents of which are incorporated by reference herein. To a solution of free base of 2-((4-(1-methyl-4-(pyridin-4-yl)-1Hpyrazol-3-yl)phenoxy)methyl)quinoline (3.0 g, 7.6 mmol) in ethyl acetate (75 ml) at 25° C., one adds 900 mg (7.6 mmol) of succinic acid in 75 ml of ethyl acetate at 25° C. The mixture is then stirred for 12 h at 25° C. The resulting precipitate is filtered, washed with diethyl ether, and dried under vacuum. Using the amounts specified the procedure yields 3.13 g of the title compound as white prism shaped crystals.

Compounds of the Formula I may have optical centers and therefore may occur in different enantiomeric and diastereomeric configurations. The present invention includes all enantiomers, diastereomers, and other stereoisomers of such compounds of the Formula I, as well as racemic compounds and racemic mixtures and other mixtures of stereoisomers thereof.

Pharmaceutically acceptable salts of the compounds of Formula I include the acid addition and base salts thereof. Suitable acid addition salts are formed from acids which form non-toxic salts. Examples include, but are not limited to, the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mandelates mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, salicylate, saccharate, stearate, succinate, sulfonate, stannate, tartrate, tosylate, trifluoroacetate and xinofoate salts. Suitable base salts are formed from bases which form non-toxic salts. Examples include, but are not limited to, the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002).

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (“glass transition”). The term “crystalline” refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (“melting point”). The compounds of the invention may also exist in unsolvated and solvated forms. The term “solvate” is used herein to describe a molecular complex comprising the compound of the invention and one or more pharmaceutically acceptable solvent molecules, for example, ethanol. The term “hydrate” is employed when the solvent is water.

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as “thermotropic” and that resulting from the addition of a second component, such as water or another solvent, is described as “lyotropic.” Compounds that have the potential to form lyotropic mesophases are described as “amphiphilic” and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

Hereinafter all references to compounds of Formula I include references to salts, solvates, multi-component complexes and liquid crystals thereof and to solvates, multi-component complexes and liquid crystals of salts thereof.

The compounds of the invention include compounds of Formula I as hereinbefore defined, including all polymorphs and crystal habits thereof, prodrugs and isomers thereof (including optical, geometric and tautomeric isomers) as hereinafter defined and isotopically-labeled compounds of Formula I.

As indicated, so-called “prodrugs” of the compounds of Formula I are also within the scope of the invention. Thus certain derivatives of compounds of Formula I which may have little or no pharmacological activity themselves can, when administered into or onto the body, be converted into compounds of Formula I having the desired activity, for example, by hydrolytic cleavage. Such derivatives are referred to as “prodrugs.” Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Prodrugs in accordance with the invention can, for example, be produced by replacing appropriate functionalities present in the compounds of Formula I with certain moieties known to those skilled in the art as “pro-moieties” as described, for example, in Design of Prodrugs by H. Bundgaard (Elsevier, 1985).

Some examples of prodrugs in accordance with the invention include, but are not limited to,

(i) where the compound of Formula I contains a carboxylic acid functionality (—COOH), an ester thereof, for example, a compound wherein the hydrogen of the carboxylic acid functionality of the compound of Formula (I) is replaced by (C₁-C₈)alkyl;

(ii) where the compound of Formula I contains an alcohol functionality (—OH), an ether thereof, for example, a compound wherein the hydrogen of the alcohol functionality of the compound of Formula I is replaced by (C₁-C₆)alkanoyloxymethyl; and

(iii) where the compound of Formula I contains a primary or secondary amino functionality (—NH₂ or —NHR where R≠H), an amide thereof, for example, a compound wherein, as the case may be, one or both hydrogens of the amino functionality of the compound of Formula I is/are replaced by (C₁-C₁₀) alkanoyl.

Further examples of replacement groups in accordance with the foregoing examples and examples of other prodrug types may be found in the aforementioned references. Moreover, certain compounds of Formula I may themselves act as prodrugs of other compounds of Formula I.

Also included within the scope of the invention are metabolites of compounds of Formula I, that is, compounds formed in vivo upon administration of the drug. Some examples of metabolites in accordance with the invention include, but are not limited to,

(i) where the compound of Formula I contains a methyl group, an hydroxymethyl derivative thereof (—CH₃→—CH₂OH):

(ii) where the compound of Formula I contains an alkoxy group, an hydroxy derivative thereof (—OR→—OH);

(iii) where the compound of Formula I contains a tertiary amino group, a secondary amino derivative thereof (—NR¹R²→—NHR¹ or —NHR²);

(iv) where the compound of Formula I contains a secondary amino group, a primary derivative thereof (—NHR¹→—NH₂);

(v) where the compound of Formula I contains a phenyl moiety, a phenol derivative thereof (13 Ph→—PhOH); and

(vi) where the compound of Formula I contains an amide group, a carboxylic acid derivative thereof (—CONH₂→COOH);

(vii) where the compound contains an aromatic nitrogen atom or an tetrtiary aliphatic amine function, an N-oxide derivative thereof.

Compounds of Formual I having a nitrogen atom in a tertiary amine functional group may be further substituted with oxygen (i.e., an N-oxide).

Compounds of Formula I containing one or more asymmetric carbon atoms can exist as two or more stereoisomers. Where a compound of Formula I contains an alkenyl or alkenylene group, geometric cis/trans (or Z/E) isomers are possible. Where structural isomers are interconvertible via a low energy barrier, tautomeric isomerism (“tautomerism”) can occur. This can take the form of proton tautomerism in compounds of Formula I containing, for example, an imino, keto, or oxime group, or so-called valence tautomerism in compounds that contain an aromatic moiety. It follows that a single compound may exhibit more than one type of isomerism.

One may use in the method of the invention all stereoisomers, geometric isomers and tautomeric forms of the compounds of Formula I, including compounds exhibiting more than one type of isomerism, and mixtures of one or more thereof. Also included are acid addition or base salts wherein the counterion is optically active, for example, d-lactate or l-lysine, or racemic, for example, dl-tartrate or dl-arginine.

Methods for making compounds used in the method of the invention are described in U.S. Pat. No. 7,429,665 and International Application Publication No. WO 2006/072828, the entire contents of both of which are incorporated herein by reference.

The term “alkyl,” as used herein, unless otherwise indicated, includes saturated monovalent hydrocarbon radicals having straight or branched moieties. Examples of alkyl groups include, but are not limited to, methyl, ethyl, propyl, isopropyl, and t-butyl.

The term “alkenyl,” as used herein, unless otherwise indicated, includes alkyl moieties having at least one carbon-carbon double bond wherein alkyl is as defined above. Examples of alkenyl include, but are not limited to, ethenyl and propenyl.

The term “alkynyl,” as used herein, unless otherwise indicated, includes alkyl moieties having at least one carbon-carbon triple bond wherein alkyl is as defined above. Examples of alkynyl groups include, but are not limited to, ethynyl and 2-propynyl.

The term “alkoxy,” as used herein, unless otherwise indicated, as employed herein alone or as part of another group refers to an alkyl, groups linked to an oxygen atom.

The term “alkylthio” as used herein, unless otherwise indicated, employed herein alone or as part of another group includes any of the above alkyl groups linked through a sulfur atom.

The term “halogen” or “halo” as used herein alone or as part of another group refers to chlorine, bromine, fluorine, and iodine.

The term “haloalkyl” as used herein, unless otherwise indicated, refers to at least one halo group, linked to an alkyl group. Examples, of haloalkyl groups include, but are not limited, to trifluoromethyl, trifluoroethyl, difluoromethyl and fluoromethyl groups. The term “cycloalkyl,” as used herein, unless otherwise indicated, includes non-aromatic saturated cyclic alkyl moieties wherein alkyl is as defined above. Examples of cycloalkyl include, but are not limited to, cyclopropyl, cyclobutyl, cyclopentyl, cyclohexyl, and cycloheptyl.

The term “aryl,” as used herein, unless otherwise indicated, includes an organic radical derived from an aromatic hydrocarbon by removal of one hydrogen, such as phenyl, naphthyl, indenyl, and fluorenyl. “Aryl” encompasses fused ring groups wherein at least one ring is aromatic.

The terms “heterocyclic,” “heterocycloalkyl,” and like terms, as used herein, refer to non-aromatic cyclic groups containing one or more heteroatoms, prefereably from one to four heteroatoms, each preferably selected from oxygen, sulfur and nitrogen. The heterocyclic groups of this invention can also include ring systems substituted with one or more oxo moieties. Examples of non-aromatic heterocyclic groups are aziridinyl, azetidinyl, pyrrolidinyl, piperidinyl, azepinyl, piperazinyl, 1,2,3,6-tetrahydropyridinyl, oxiranyl, oxetanyl, tetrahydrofuranyl, tetrahydrothienyl, tetrahydropyranyl, tetrahydrothiopyranyl, morpholino, thiomorpholino, thioxanyl, pyrrolinyl, indolinyl, 2H-pyranyl, 4H-pyranyl, dioxanyl, 1,3- dioxolanyl, pyrazolinyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, quinolizinyl, quinuclidinyl, 1,4-dioxaspiro[4.5]decyl, 1,4-dioxaspiro[4.4]nonyl, 1,4- dioxaspiro[4.3]octyl, and 1,4-dioxaspiro[4.2]heptyl.

The term “heteroaryl,” as used herein, refers to aromatic groups containing one or more heteroatoms (preferably oxygen, sulfur and nitrogen), preferably from one to four heteroatoms. A multicyclic group containing one or more heteroatoms wherein at least one ring of the group is aromatic is a “heteroaryl” group. The heteroaryl groups of this invention can also include ring systems substituted with one or more oxo moieties. Heteroaryl groups containing a tertiary nitrogen may also be further substituted with oxygen (i.e., an N-oxide).

Examples of heteroaryl groups are pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazoiyl, isothiazolyl, pyrrolyl, indolyl, benzimidazolyl, benzofuranyl, cinnolinyl, indazolyl, indolizinyl, phthalazinyl, triazinyl, isoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl. For clarity, the term heteroaryl includes the heteroaryl structure in substituent Z in Formula I (i.e., the heteroaryl structure containing Y).

Unless otherwise indicated, the term “one or more” substituents, or “at least one” substituent as used herein, refers to from one to the maximum number of substituents possible based on the number of available bonding sites.

Unless otherwise indicated, all the foregoing groups derived from hydrocarbons may have up to about 1 to about 20 carbon atoms (e.g. C₁-C₂₀ alkyl, C₂-C₂₀ alkenyl, C₃-C₂₀ cycloalkyl, 3-20 membered heterocycloalkyl; C₆-C₂₀ aryl, 5-20 membered heteroaryl, etc.) or 1 to about 15 carbon atoms (e.g., C₁-C₁₅ alkyl, C₂-C₁₅ alkenyl, C₃-C₁₅ cycloalkyl, 3-15 membered heterocycloalkyl, C₆-C₁₅ aryl, 5-15 membered heteroaryl, etc.) , or 1 to about 12 carbon atoms, or 1 to about 8 carbon atoms, or 1 to about 6 carbon atoms.

### Formulation and Administration

The compound of the invention may be administered via either the oral, transdermal (e.g. through the use of a patch), intranasal, sublingual, rectal, parenteral, or topical routes. In one embodiment, the compound is delivered by injecting it into the eye; topical administration is unlikely to achieve a dose that is high enough for the compound to be effective in treating disorders of the retina.

In one embodiment the compound is administered at doses ranging from about 0.25 mg up to about 1500 mg per day; in another embodiment the compound is administered at doses of 0.25 to about 300 mg per day in single or divided doses; in another embodiment the compound is administered at doses of 0.01 mg to about 10 mg per kg of body weight per day, although variations will necessarily occur depending upon the weight and condition of the subject being treated and the particular route of administration chosen, as well as the individual's responses to the treatment, the formulation chosen, and the length of time the patient is treated. In some instances, doses less than 0.25 mg per day may be adequate, while in other cases still larger doses may be employed without causing any harmful side effects, provided that such larger doses are first divided into several small doses for administration throughout the day.

The active compounds can be administered alone or in combination with pharmaceutically acceptable carriers or diluents by any of the several routes previously indicated. More particularly, the active compounds can be administered in a wide variety of different dosage forms, e.g., they may be combined with various pharmaceutically acceptable inert carriers in the form of tablets, capsules, transdermal patches, lozenges, troches, hard candies, powders, sprays, creams, salves, suppositories, jellies, gels, pastes, lotions, ointments, aqueous solutions, aqueous suspensions, injectable solutions, elixirs, syrups, and the like. Such carriers include solid diluents or fillers, sterile aqueous media and various non-toxic organic solvents. In addition, oral pharmaceutical compositions can be suitably sweetened and/or flavored. In general, the active compounds are present in such dosage forms at concentration levels ranging from about 5.0% to about 70% by weight.

For oral administration, tablets containing various excipients such as microcrystalline cellulose, sodium citrate, calcium carbonate, dicalcium phosphate and glycine may be employed along with various disintegrants such as starch (preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc can be used for tabletting purposes. Solid compositions of a similar type may also be employed as fillers in gelatin capsules; preferred materials in this connection also include lactose or milk sugar, as well as high molecular weight polyethylene glycols. When aqueous suspensions and/or elixirs are desired for oral administration the active ingredient may be combined with various sweetening or flavoring agents, coloring matter and, if so desired, emulsifying and/or suspending agents, together with such diluents as water, ethanol, propylene glycol, glycerin and various combinations thereof.

For parenteral administration, a solution of an active compound in a pharmaceutically acceptable oily or aqueous vehicle such as but not limited to sesame oil, peanut oil or aqueous propylene glycol, can be employed. The aqueous solutions should be suitably buffered, if necessary, and the liquid diluent first rendered isotonic.

The preparation of the solutions is under sterile conditions and is readily accomplished by standard pharmaceutical techniques well known to those skilled in the art. Parenteral administration may be by injection, including the intravenous, intraarticular, intramuscular, and subcutaneous forms. The aqueous solutions are suitable for intravenous injection purposes. The oily solutions are suitable for intraarticular, intramuscular and subcutaneous injection purposes.

It is also possible to administer the active compounds topically and this can be done by way of creams, a patch, jellies, gels, pastes, ointments and the like, in accordance with standard pharmaceutical practice.

### EXAMPLES

The invention is illustrated by the following example. The compound of the invention was tested with the following experimental protocol:

(1) Mice are dosed twice daily at 10 mg/kg or vehicle, IP.

(2) On day 3, mice are exposed to 10,000 lux light for 30 min, 2 h after the first dosing and 6 h before the last dosing.

(3) On day 11 ERG measurements are taken.

(4) On day 12, mice are sacrificed for retinal histology.

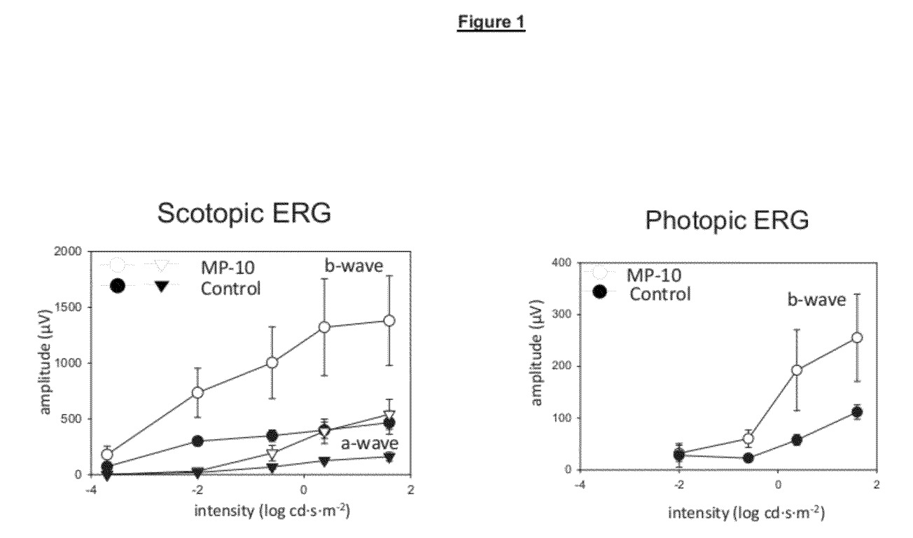

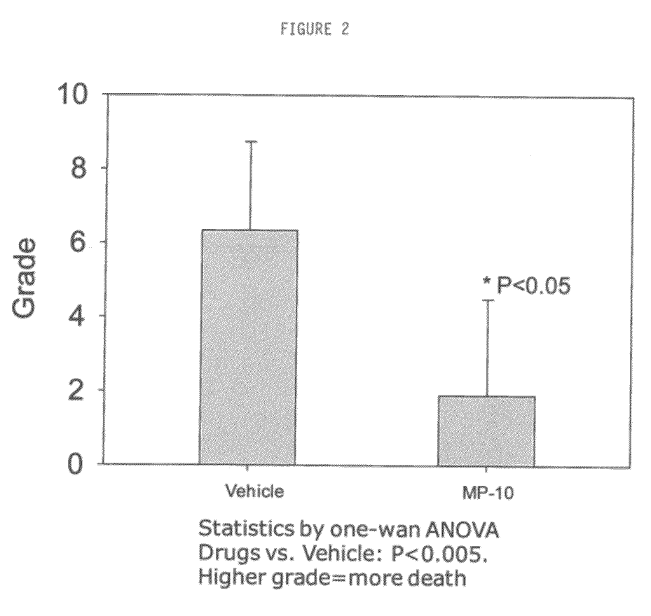

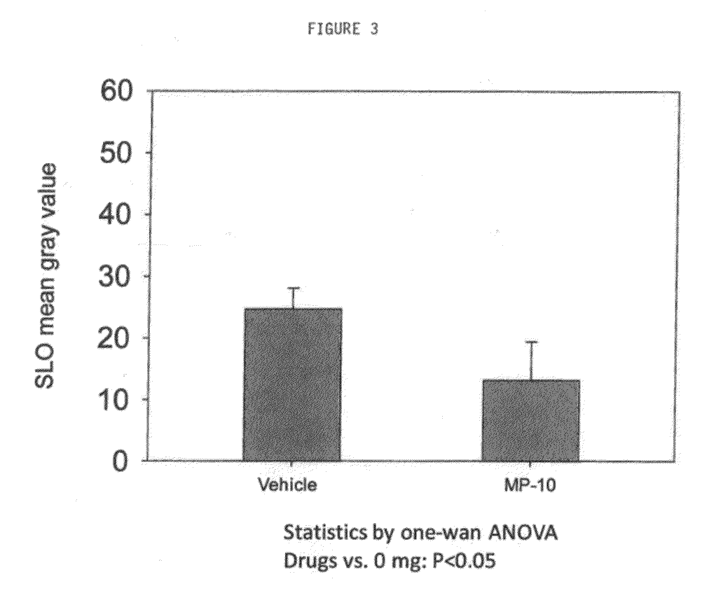

The ERG functional assay measures the functional integrity of retina. In this assay drug treated mice showed very little loss of function while the vehicle treated mice showed severe loss of function (FIG. 1). In addition, photoreceptor cells were also strongly protected in this study as shown in FIG. 2. In the model, autofluorescence appears as the retina degenerates. FIG. 3 shows that the compound of the invention can reduce this autofluorescence.

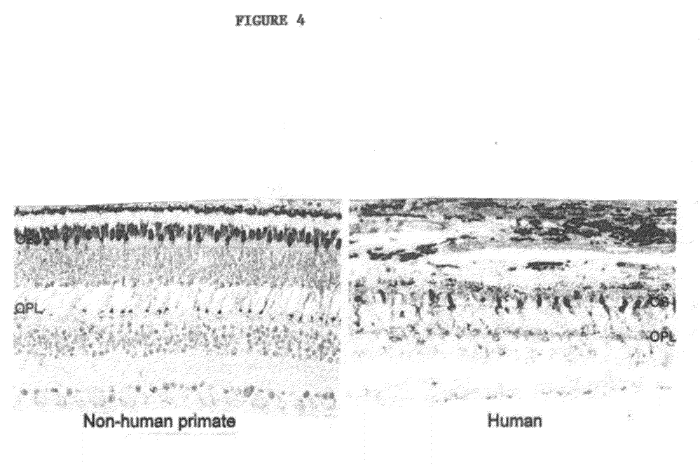

In immunohistochemistry studies in rabbit, non-human primate and human retinas, the inventors demonstrated that PDE10A protein is expressed in photoreceptors and RPE/choroid (FIG. 4).

These data confirm the utility of the compounds of the invention in treating retinal diseases.

Each and every reference (whether patent publication or a scientific/journal publication) disclosed herein is incorporated by reference herein for all purposes.

The details of specific embodiments described in this invention are not be construed as limitations. Various equivalents and modifications may be made without departure from the essence and scope of this invention, and it is understood that such equivalent embodiments are part of this invention.

What is claimed is:

1. A method for treating a disorder of the retina, the method comprising administering to a patient in need thereof, a therapeutically effective amount of a compound of Formula I: 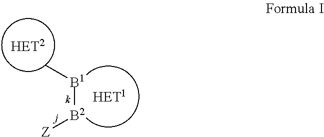
or a pharmaceutically acceptable salt thereof, wherein:
Z is 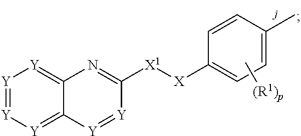
R¹ is each independently selected from a group consisting of hydrogen, halogen, hydroxyl, cyano, C₁ to C₈ alkyl, C₂ to C₈ alkenyl, C₂ to C₈ alkynyl, C₁ to C₈ alkoxy, C₁ to C₈ haloalkyl, C₃ to C₈ cycloalkyl, C₃ to C₈ cycloalkyl-C₁ to C₈ alkyl, 4 to 7 membered heterocycloalkyl, C₁ to C₈ alkylthio, —NR³R³, —O—CF₃, —S(O)ₙ13 R³, C(O)—NR³R³, and C₁ to C₈ alkyl substituted with a heteroatom wherein the heteroatom is selected from a group consisting of nitrogen, oxygen and sulfur and wherein the heteroatom may be further substituted with a substituent selected from a group consisting of hydrogen, C₁ to C₈ alkyl, C₃ to C₈ cycloalkyl, C₂ to C₈ alkenyl, C₂ to C₈ alkynyl, and C₁ to C₈ haloalkyl;
each R³ is independently selected from a group consisting of hydrogen, C₁ to C₈ alkyl, C₂ to C₈ alkenyl, C₂ to C₈ alkynyl, C₁ to C₈ haloalkyl, and C₃ to C₈ cycloalkyl;
R² is selected from the group consisting of hydrogen, C₁ to C₈ alkyl, C₃ to C₈ cycloalkyl-C₁ to C₈ alkyl, C₂ to C₈ alkenyl, C₂ to C₈ alkynyl, C₁ to C₈ haloalkyl and C₃ to C₈ cycloalkyl;
HET¹ is selected from a group consisting of a monocyclic heteroaryl and a bicyclic heteroaryl, wherein the monocyclic and bicyclic heteroaryl may be optionally substituted with at least one R⁴;
R⁴ is selected from a group consisting of halogen, hydroxyl, cyano, C₁ to C₈ alkyl, C₂ to C₈ alkenyl, C₂ to C₈ alkynyl, C₁ to C₈ alkoxy, C₃ to C₈ cycloalkyl, C₃ to C₈ cycloalkyl-C₁ to C₈ alkyl, C₁ to C₈ alkylthio, and C₁ to C₈ alkyl substituted with a substituent is selected from the group consisting of —OR⁸, —NR⁸R⁸, and —SR⁸, wherein R⁸ is independently selected from the group consisting of hydrogen and C₁ to C₈ alkyl;
HET² is a monocyclic or bicyclic heteroaryl, wherein the monocyclic and bicyclic heteroaryl optionally substituted with at least one R⁵, with the proviso that HET² is not tetrazole;
R⁵ is independently selected from a group consisting of halogen, hydroxyl, cyano, C₁ to C₈ alkyl, C₂ to C₈ alkenyl, C₂ to C₈ alkynyl, C₁ to C₈ alkoxy, C₃ to C₈ cycloalkyl, C₃ to C₈ cycloalkyl-C₁ to C₈ alkyl, C₁ to C₈ alkylthio, —NR⁷R⁷ and C₁ to C₈ haloalkyl;
B¹ and B² are adjacent atoms in Het¹ which are independently selected from a group consisting of carbon and nitrogen;
bond j is a covalent bond between Z and B²;
bond k is a covalent bond in Het¹ between B¹ and B²;
X and X¹ are each independently selected from the group consisting of oxygen, sulfur, C(R₂)₂ and NR₂; provided that at least one of X or X¹ is carbon;
Y is selected from a group consisting of carbon and nitrogen, provided that when Y is carbon it is substituted with R⁶;
wherein each R⁶ is independently selected from a group consisting of hydrogen, halogen, hydroxyl, cyano, C₁ to C₈ alkyl, C₂ to C₈ alkenyl, C₂ to C₈ alkynyl, C₁ to C₈ alkoxy, C₁ to C₈ cycloalkyl, C₃ to C₈ cycloalkyl-C₁ to C₈ alkyl, C₁ to C₈ alkylthio, C₁ to C₈ haloalkyl, —NR⁷R⁷, —O—CF₃, —S(O)m—R⁷, and C(O)—NR⁷R⁷, C₁ to C₈ alkyl substituted with a heteroatom wherein the heteroatom is selected from a group consisting of nitrogen, oxygen and sulfur and wherein the heteroatom may be further substituted with a substituent selected from the group consisting of hydrogen, C₁ to C₈ alkyl, C₃ to C₈ cycloalkyl, C₂ to C₈ alkenyl, C₂ to C₈ alkynyl, and C₁ to C₈ haloalkyl; and
wherein each R⁷ is independently selected from the group consisting of hydrogen and C₁-C₈ alkyl; p is 1, 2 or 3; n is 0, 1 or 2; and m is 0, 1 or 2.

2. The method of claim 1, wherein HET¹ is a 5 membered heteroaryl group.

3. The method of claim 1, HET¹ is selected from the group consisting of pyrazole, isoxazole, triazole, oxazole, thiazole and imidazole.

4. The method of claim 1, wherein HET² is selected from the group consisting of 4-pyridyl, 4- pyridazine and isoxazole.

5. The method of claim 1, wherein HET² is 4-pyridyl.

6. The method of claim 1, wherein the compound is selected from the group consisting of: 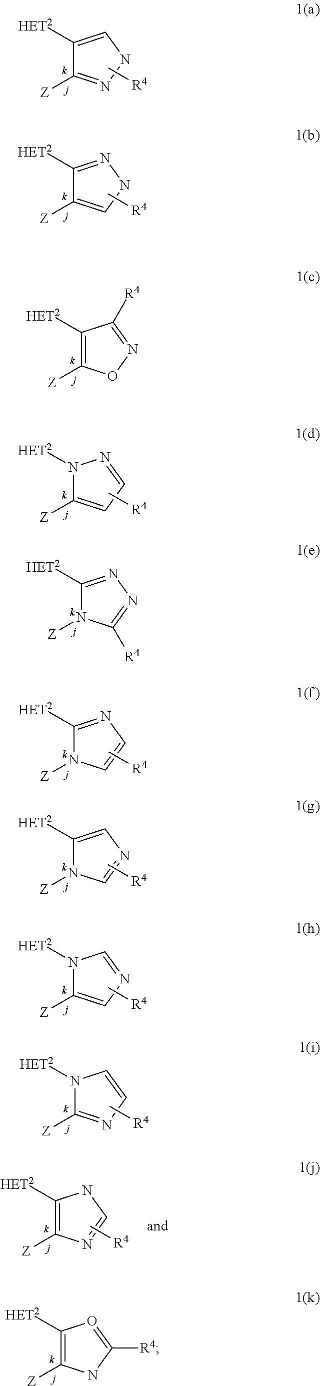
or a pharmaceutically acceptable salt thereof.

7. The method of claim 1, wherein the compound has the structure 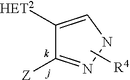
or a pharmaceutically acceptable salt thereof.

8. The method of claim 1, wherein the compound has the structure 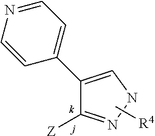
or a pharmaceutically acceptable salt thereof.

9. The method of claim 1, wherein Y is selected from the group consisting of carbon and nitrogen, provided that not more than one Y is nitrogen.

10. The method of claim 1 wherein X¹ is carbon and X is oxygen,

11. The method of claim 1, wherein all Y's are carbon.

12. The method of claim of 1, wherein the compound has the structure 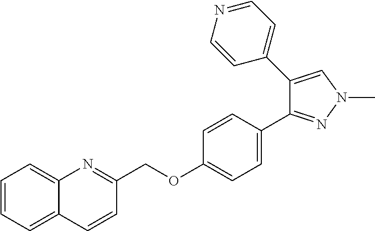
or a pharmaceutically acceptable salt thereof.

13. The method of claim 1, wherein the compound is administered as the succinate salt.

14. The method of claim 1, wherein the retinal disease is selected from the group consisting of age related macular degeneration, retinitis pigmentosa, Stargardt's disease and other retinal dystrophies, macular edema, retinal detachment, retinal trauma, retinal tumors and retinal diseases associated with them, congenital hypertrophy of the retinal pigmented epithelium, acute posterior multifocal placoid pigment epitheliopathy, and acute retinal pigment epithelitis.

15. The method of claim 1, wherein the compound is administered orally or by injecting it into the eye.
